(19) United States
(12) Patent Application Publication
(10) Pub. No.: US 2013/0178419 A1
(43) Pub. Date: Jul. 11, 2013

(54) METHOD OF TREATING PAIN BY ADMINISTRATION OF NERVE GROWTH FACTOR

(71) Applicant: Beech Tree Labs, Inc., Delanson, NY (US)

(72) Inventor: John McMichael, Delanson, NY (US)

(73) Assignee: Beech Tree Labs, Inc., Delanson, NY (US)

(21) Appl. No.: 13/735,273

(22) Filed: Jan. 7, 2013

Related U.S. Application Data
(60) Provisional application No. 61/583,538, filed on Jan. 5, 2012.

(51) Int. Cl. A61K 38/18

(52) U.S. Cl. CPC A61K 38/185
USPC 514/8.4

(57) ABSTRACT

The invention provides methods and compositions for treatment of acute and chronic pain comprising the administration of Nerve Growth Factor.

### CROSS-REFERENCE TO RELATED APPLICATION

This application claims the benefit of priority to U.S. Provisional Application No. 61/583,538, filed Jan. 5, 2012, the disclosure of which is incorporated herein by reference in its entirety.

### BACKGROUND OF THE INVENTION

Nerve growth factor (NGF), a prototypical neurotrophic factor and member of the neurotrophin family, promotes a wide range of responses in target cells. These responses include, but are not limited to, neuron differentiation, maintenance of neuronal survival, and regulation of metabolic activities. Nerve growth factor is well-characterized neurotrophic factor that is essential for the normal development and function of basal forebrain cholinergic neurons in the central nervous system (CNS) (Ghahn et al., 1983; Thoenen and Edgar, 1985). A central area of research in application of nerve growth factor has been its application to age-related cognitive impairments due to the atrophy or loss of basal forebrain cholinergic neurons (Armstrong et al., Neurobiol. Aging 14:457-470 (1993)). For example, studies have shown the intraventricular infusion of NGF can reduce cholinergic neuron atrophy and improve spatial learning or memory retention in aged rats (Scali et al., Neurosci Lett 170:117-120 (1994); Markowska et al., J. Neurosci 14:4815-4825 (1994)). Due to studies indicating decreased immunoreactivity for the NGF receptor in basal forebrain of aged rodents, neural growth factor appears to be linked to spatial learning and memory retention (Fischer et al., Neurobiol. Aging 13:9-23 (1992)). One example of therapeutic uses of NGF includes administering NGF to patients with senile dementia of the Alzheimer's type (SDAT). The problem of such treatment is NGF does not pass through the blood-brain barrier in physiologically relevant amounts and treatments required intracranial surgery (Kordower et al., Exp. Neurol. 124:21-30 (1993). Novel carrier systems consisting of NGF covalently linked to an anti-transferrin receptor antibody (OX-26) have been able to cross the blood-brain barrier.

Of interest to the present application are the disclosures in US 2004/0127409 in which administration of nerve growth factor is disclosed as being useful for the treatment of psychological conditions including aspects of depression. Specifically, disclosed are methods for treating patients with symptoms of major depression by topically, sublingually, or subcutaneously administering a small amount of nerve growth factor. Such methods were disclosed to be useful for treating dysthymia including, but not limited to, treating the symptoms of distress and difficulty in performing everyday functions. Methods of the invention are also useful for treating depressed moods including, but not limited to, elderly depression and adolescent depression. In those cases, methods of the invention reduce feelings of sadness, gloominess, emptiness, fatigue, loss of appetite, body aches and pains, and sleeping difficulties. Also disclosed are treatment of panic disorder (chest pain) and pms (pain including headache, backache, menstrual cramping, and breast tenderness.)

Also of interest to the present invention are the disclosures of US 2007/0270347 which is directed to the administration of NGF for treatment of constipation and of US 2010/093631 which is directed to the administration of NGF for the treatment of cravings.

Also of interest to the present invention is the disclosure of US 2010/0291083 which is directed to treatment of chronic pain by the administration of an anti-TrkA antibody capable of inhibiting the binding between NGF and its high affinity receptor TrkA.

Of further interest to the present invention is the disclosure of US 2011/0033447 which is directed to the use of anti-NGF antibodies including anti-NGF antagonist antibodies for the treatment and prevention of post-surgical pain, rheumatoid arthritis pain and osteoarthritis pain.

Despite these recent applications of nerve growth factor, there remains a desire to use NGF to remedy other disorders including acute and chronic pain.

### BRIEF SUMMARY OF THE INVENTION

The present invention provides methods of treating pain comprising administering to a subject in need thereof an effective amount of a composition comprising Nerve Growth Factor (NGF). The methods may be used to treat both acute and chronic pain and in particular is useful for the treatment of pain associated with arthritis including osteoarthritis and pain associated with spondylarthritis.

Nerve growth factor is generally administered at lower levels of than in previous therapies as it is believed that higher levels of administration may increase anxiety. The nerve growth factor is preferably administered at a dosage of from about 0.001 to 1 microgram per day with dosages of from 0.01 to 0.1 microgram per day being preferred. The nerve growth factor may be administered by a variety of modes including from the group consisting of sublingual, bucal, oral drench, subcutaneous, intradermal, and intravenous with sublingual administration being particularly preferred.

The Nerve growth factor holoprotein may be administered but can also be administered as the beta-subunit.

The invention also provides a pharmaceutical composition for treating pain comprising Nerve Growth Factor in an amount effective to treat said pain which can optionally further comprise a pharmaceutically acceptable carrier, excipient or diluent. Preferred pharmaceutical compositions include those wherein the Nerve Growth Factor is present in an amount of from 0.001 to 1 microgram or more preferably wherein the Nerve Growth Factor is present in an amount of from 0.01 to 0.1 microgram.

### DETAILED DESCRIPTION OF THE INVENTION

The invention is directed to methods of treating pain by the administration of an effective amount of nerve growth factor (NGF) or a subunit thereof and particularly the beta-subunit of NGF. Nerve growth factor is commercially available from suppliers such as Sigma. Particularly preferred is the use of recombinant produced beta-subunit of NGF which is available from Sigma and EMD Biosciences. The nerve growth factor is preferably administered in an amount ranging from about 0.001 to 10 microgram per day and is preferably formulated in a liquid vehicle and provided at a concentration of approximately 0.04 micrograms as a single drop. A single drop of nerve growth factor is within the range of 0.001 to 1 microgram. More preferably, a drop of nerve growth factor composition is in the amount of 0.02 micrograms per drop. The nerve growth factor composition is more preferably administered in an amount ranging from about 0.05 to 1 microgram per day or even more preferably administered in an amount ranging from about 0.01 to 0.1 micrograms per day. A preferred route of administration is sublingual, but other routes, such as bucal, oral drench, subcutaneous, intradermal, and intravenous, are expected to work.

The following Examples illustrate the methods of the invention with respect to treatment of various pain conditions. Numerous improvements and further aspects of the invention are apparent to the skilled artisan upon consideration of the Examples which follow.

### Example 1

According to this example, a patient complaining of pain following oral surgery was advised to take one drop of an NGF solution (0.05 ml) (0.008 μg/drop) of NGF by mouth every 15 minutes until the pain was relieved. The patient took first drop at t₀ and second at t₁₅ minutes but a third drop was not necessary as the pain was relieved.

### Example 2

According to this example, a patient suffering knee pain from an osteoarthritis-type condition was advised to take one drop of an NGF solution (0.05 ml) (0.008 μg/drop) of NGF by mouth every 15 minutes until the pain was relieved. Pain was relieved after the third drop (t-30 minutes).

In both Examples 1 and 2 the patients were advised to use additional NGF drops on an as needed basis. Followup of the two patients by a Registered Nurse reported the patients taking one drop very infrequently, if at all.

### Example 3

According to this example, a teenage subject was treated for pain in his right lateral buttock thought to be a weak gluteous medius muscle. He had physical therapy and completed an exercise program which led to several weeks without pain. He later developed pain in the right groin and hip during cross country running season. This pain eased during the winter but returned during track season. An X-ray of his sacrolliac joints confirmed a right sacroilitis which was later diagnosed as HLA-27 positive spondyloarthritis by a rheumatologist at Children's Hospital of Pittsburgh. Over the next five years, his pain was controlled with conservative treatment and light exercise. More recently the subject's pain and spasm activity increased especially in the morning. Treatment by sublingual administration of NGF, was added to his treatment at 1 drop sublingual daily or as needed which was typically one drop every two or three days. His pain improved and this treatment was eventually discontinued. (It was noted that when the patient overdosed on the NGF by taking a drop too frequently, he becomes anxious for a period of several hours.)

More recently, the subject's pain returned and responded again to the addition of NGF at one drop (0.05 ml) (0.008 μg/drop) daily or twice a day. The subject reported that he felt as if his back spasm eased as the pain left. He has returned to exercising and he is focusing better during his medical school studies with lessening of pain.

### Example 4

According to this example, a 60 year old female patient with periodontal deterioration due to two root canal breakdowns from screws used many years before, causing gum erosion, a fistula from gums to sinus cavity, with inflammation, toxins and infection. More recently, scaling and root planing were performed by a periodondist. Instead of using traditional pharmaceutical therapy of Triazolam 0.25 mg, for relaxation for a 2 hour procedure of Scaling and Root Planing, and narcotics (Hydrocodone 5 mg), for post procedure pain, NGF drops were used at 2 drops (0.05 ml) (0.008 μg/drop), ½ hour before the procedure, with 2 tabs of an anxiolytic supplement containing calcium and magnesium taurates and cobamamide-co-enzyme B12. (Shpilkes).

Following the procedure, additional NGF drops were administered every 3 hours while awake to prevent pain or inflammation. When the anesthetic (Novocaine) wore off, there was no pain.

### Example 5

According to this example, a 61-year old female patient with fibromyalgia characterized by nearly al tender point associated with this condition. The patient was started on NGF at one drop twice a day. Doses varied from 1 to 4 sublingual drops of NGF daily. Her tender points have lessened and overall her pain is much less than prior to treatment with the NGF. She noted that as he pain eases, her overall mood improves. No side effects were observed with the NGF treatment.

### Example 6

According to this example, a patient with demyelinating peripheral neuropathy has near complete resolution of lower extremity paresthesias with the first two weeks of treatment with 1 to 4 drops of NGF administered sublingually daily.

### Example 7

According to this example, a patient with polymyalgia rheumatic noticed significant improvement in fatigue, shoulder and arm pain within two weeks of starting treatment with 1 to 4 drops of NGF administered sublingually daily.

The foregoing Examples demonstrate that treatment with NGF positively impacted subjects suffering from both inflammatory and non-inflammatory pain. No side effects were observed and the pain relieving effects remained for several years following treatment.

Numerous modifications and variations in the practice of the invention are expected to occur to those skilled in the art upon consideration of the presently preferred embodiments thereof. Consequently, the only limitations which should be placed upon the scope of the invention are those which appear in the appended claims.

What is claimed is:

1. A method of treating pain comprising administering to a subject in need thereof an effective amount of a composition comprising Nerve Growth Factor.

2. The method of claim 1 wherein the pain is acute pain.

3. The method of claim 1 wherein the pain is chronic pain.

4. The method of claim 1 wherein the pain is associated with arthritis.

5. The method of claim 4 wherein the pain is associated with osteoarthritic.

6. The method of claim 4 wherein the pain is associated with spondylarthritis.

7. The method of claim 1, wherein said Nerve Growth Factor is administered by a mode selected from the group consisting of sublingual, bucal, oral drench, subcutaneous, intradermal, and intravenous.

8. The method of claim 3, wherein said Nerve Growth Factor is administered sublingually.

9. The method of claim 1, wherein said Nerve Growth Factor is administered at a daily dosage of from 0.001 to 1 microgram per day.

10. The method of claim 1, wherein said Nerve Growth Factor is administered at a daily dosage of from 0.01 to 0.1 microgram per day.

11. The method of claim 1 wherein the Nerve Growth Factor is a beta-subunit of Nerve Growth Factor.

12. A pharmaceutical composition for treating pain comprising Nerve Growth Factor in an amount effective to treat said pain.

13. The pharmaceutical composition of claim 12, wherein the composition further comprises a pharmaceutically acceptable carrier, excipient or diluent.

14. The pharmaceutical composition of claim 12 wherein the Nerve Growth Factor is present in an amount of from 0.001 to 1 microgram.

15. The pharmaceutical composition of claim 12 wherein the Nerve Growth Factor is present in an amount of from 0.01 to 0.1 microgram.
